(19) United States
(12) Patent Application Publication
(10) Pub. No.: US 2023/0263754 A1
(43) Pub. Date: Aug. 24, 2023

(54) SOLID DISPERSIONS AND PARTICLES AND METHODS FOR CONTROLLED-RELEASE OF LIPID-SOLUBLE OR DISPESIBLE ACTIVES

(71) Applicant: YISSUM RESEARCH DEVELOPMENT COMPANY OF THE HEBREW UNIVERSITY OF JERUSALEM LTD., Jerusalem (IL)

(72) Inventor: Abraham Jacob DOMB, Jerusalem (IL)

(21) Appl. No.: 17/996,538

(22) PCT Filed: Apr. 21, 2021
(86) PCT No.: PCT/IL2021/050452

§ 371 Date: Oct. 19, 2022

Related U.S. Application Data
(60) Provisional application No. 62/704,096, filed on Apr. 21, 2020.

(51) Int. Cl. A61K 31/192; A61K 31/343; A61K 31/167; A61K 31/12; A61K 9/10; A61K 9/50; A61K 9/48; A01N 25/28; A01N 43/40

(52) U.S. Cl. CPC A61K 31/192; A01N 25/28; A01N 43/40; A61K 9/10; A61K 9/501; A61K 9/4875; A61K 9/5015; A61K 9/5036; A61K 9/5084; A61K 31/12; A61K 31/167; A61K 31/343

(57) ABSTRACT

The invention provides a particle comprising a lipid core and surface-decorated (nano)particles of at least one stabilizing material, for delivery of active agents.

### TECHNOLOGICAL FIELD

The invention generally concerns particulate formulations for delivery of lipid-soluble or lipid dispersible active agents.

### BACKGROUND

Pickering emulsions are known since 1907. These are oil in water emulsions, where liquid oils are dispersed in aqueous media, with the dispersing agents separating the oil and water are not molecular emulsifiers, but rather amphiphilic solid nanoparticles that cover the surface of the oil droplet that allows oil dispersion in water. The amphiphilic nanoparticles are typically colloidal silica, hydroxyapatite, clay, cellulose or amylose crystals, magnetic nanoparticles, carbon nanotube, metal oxides, lipids or polymeric nanoparticles with amphiphilic surfaces.

There have been numerous reports on the use of Pickering emulsions for the delivery of drugs, agriculture agents, catalysts, cosmetics, food additives with applications is a broad range of medical and non-medical fields.

However and despite the fact that Pickering emulsions, having lipid liquid cores at room temperature, have been subject of continuous research for over 100 years, these dispersions suffer from instability when exposed to high temperature, salts, physical forces, or when standing over time. Publication [16] describes lipid microparticles of 400 microns containing ibuprofen, where silica nanoparticles are used to prepare the microparticles and are dispersed in the lipid core which affects release of the ibuprofen from these particles. In another publication [17] a solid paraffin particles having surface nanoparticles is described for the preparation Janus nanoparticles.

### BACKGROUND ART

Chinese Patent Application No. CN110755383
Chinese Patent Application No. CN110003498
Chinese Patent Application No. CN109222179
International Patent Publication No. WO20027722
International Patent Publication No. WO20007885
International Patent Publication No. WO18104642
International Patent Publication No. WO15170099
International Patent Publication No. WO15160794
US Patent No. 9,388,335
US Patent No. 9,867,763
International Patent Publication No. WO12082065
International Patent Publication No. WO08138894
European Patent No. 1477152
International Patent Publication No. WO03049710
European Patent No. 0987002.
International J. of Pharmaceutics, 422 (2012) 56-67.
Langmuir 2006, 22, 9495-9499.

### GENERAL DESCRIPTION

In an attempt to overcome limitations of the art, the inventors of the technology disclosed herein have developed a platform for the facile and effective delivery of lipid-soluble or dispersible active and non-active agents. The lipid-soluble or dispersible agents are formulated into solid particles that are surface-decorated with a plurality of particles of a different material, which act as a dispersing material; thereby enabling dispersion of the solid particles in aqueous media. The agent to be delivered is typically contained in the solid particles, but may also or alternatively be contained in or associated with the particles decorating the particles surface. A depiction of a delivery system according to the invention is provided in Scheme 1 below.

[image]

### Scheme 1

As depicted in Scheme 1, the particle of the invention has a core comprising a solid lipid and a plurality of (nano)particles that decorate the particle circumference. These (nano)particles stabilize the lipid cores by adsorbing at the water-lipid (or liquid-solid) interface, thereby forming a barrier around the lipid cores so that contacting or neighboring cores are not able to coalesce. The effectiveness of the decorating particles in stabilizing formulations or emulsions containing the particles depends, inter alia, on particle size, particle shape, particle concentration, particle wettability and the interactions between particles. The (nano)particles are selected to be of a small enough size, so that they can coat the surfaces of the lipid cores and further to have sufficient affinity for both the water and lipid phases; thereby rendering the (nano)particles effective in forming continuous and disperse phases and stabilizing the dispersion. The decorating (nano)particles have a diameter that is up to 10% of the core particle size.

The degree of decoration by the (nano)particles (i.e., the number of (nano)particles per surface area, or density of nanoparticles on the surface) may be varied to tailor desired formulation properties.

Thus, in a first aspect, the invention provides a particle comprising a lipid core (e.g., in a form of a solid wax), the circumference of the core being surface-decorated with a plurality of (nano)particles of at least one stabilizing material, the lipid core being an active material or comprising at least one lipid-soluble or lipid-dispersible active material and wherein (at least a portion of) the (nano)particles optionally comprise at least one active material or wherein the (nano)particles are optionally associated with at least one active material. The active material of the core or contained in the core, and the active material contained in the (nano)particles or associated thereto may be the same or different.

The invention further provides a particle comprising a lipid core, the circumference of the core being surface-decorated with a plurality of (nano)particles of at least one stabilizing material, the lipid core being an active material or comprising at least one lipid-soluble or lipid-dispersible active material, wherein the lipid core is free (or essentially or substantially free) of nanoparticles, and wherein the plurality of (nano)particles optionally comprise or are optionally associated to at least one active material.

The invention further provides a particle comprising a lipid core, the circumference of the core being surface-decorated with a plurality of (nano)particles of at least one stabilizing material, the lipid core being an active material or comprising at least one lipid-soluble or lipid-dispersible active material, wherein the (nano)particles comprise or are associated to at least one active material, and wherein the active material of the core or contained in the core, and the active material contained in or associated to the (nano)particles are the same or different.

The invention further provides a dispersion comprising water or an aqueous medium and a plurality of particles according to the invention.

The particles of the invention are structured and composed of materials permitting improved delivery of one or two or more types of active or non-active agent(s). In some embodiments, the particles comprise an active material or a lipid-soluble material or a lipid-dispersible material (active or non-active), in the particles’ lipid cores, and optionally non-lipidic agents (active or non-active) in the surface-decorating (nano)particles, permitting sequential or synchronized delivery of the agents. As noted herein, the (nano)particles may comprise at least one active material or may be associated with such a material. The association of the (nano)particles to the active material may be a chemical association or a physical association. In other words, association may be by:
Impregnation of the active material that is embedded in the (nano)particles;
absorption or adsorption of the active material to the surface of the (nano)particle;
surface binding by covalent or electrostatic or hydrogen bonds of the active material to the surface of the (nano)particles. Such surface binding may be direct or indirect through conjugation of functional groups such as amino or carboxylic acid groups or vinyl groups that allow polymerization and binding the active materials thereto.

The surface of the (nano)particles may additionally or alternatively be associated with polymeric or other functional materials that modify properties of the particles of the invention, as disclosed herein.

In particles of the invention, where the decorating (nano)particles comprise or are associated with an active or non-active material, not all (nano)particles need to comprise or be associated with the active or non-active material. In some embodiments, the full (nano)particle population decorating a single particle or a majority of the (nano)particles comprises or is associated with the active or non-active material. In other embodiments, only a portion of the (nano)particles comprises or is associated with an active or non-active material. The portion may be between 1 and 99% of the (nano)particles present on the circumference of the particle. In some embodiments, the term “portion” means between 1 and 10%, 1 and 15%, 1 and 20%, 1 and 25%, 1 and 30%, 1 and 35%, 1 and 40%, 1 and 45%, 1 and 50%, 1 and 55%, 1 and 60%, 1 and 65%, 1 and 70%, 1 and 75%, 1 and 80%, 1 and 85%, or 1 and 90% of the (nano)particles present on the circumference of the particle.

The lipid-core is typically, but not necessarily, provided as a material particle having a size ranging from 0.3 to 200 microns, Where methods for preparing particles of the invention make use of dissolution steps or other processing steps that alter the shape, size and form of the lipid material, when formed into particles of the invention, the lipid material typically arranges into spherical particles, hence lipid-cores, of sizes which may be tailored and controlled for a particular use. In some embodiments, the lipid-core is a microparticle having a size ranging between 1 and 200 microns. In some embodiments, a population of particles according to the invention comprises a mixture of different lipid-cores; some are nanoparticles in size and others are microparticles in size.

The lipid-core is typically free of particles, e.g., nanoparticles. In some cases, the lipid-core is essentially or substantially free of nanoparticles, as further defined herein. In such a case, the maximum amount of nanoparticles in the lipid-core does not exceed a functional amount or does not exceed ppm amounts. Impurity amounts of nanoparticles may be contained in the lipid-core, wherein such amounts are not functional or do not contribute to the function or characterization of the particle as a whole.

The density of the (nano)particles decorating the surface of the lipid particles, i.e., lipid core does not typically exceed 15%. In other words, in a typical system of the invention, not more than 15%, 14%, 13%, 12%, 11%, 10%, 9%, 8%, 7%, 6% or 5% of the circumference of the lipid core is associated with (nano)particles. Notwithstanding, the distribution of (nano)particles, inhomogeneous or homogeneous, as may be the case, is sufficient to prevent the cores from coalescing. In some cases, to achieve stabilization of the lipid cores and prevent particles from coalescing, the distribution of the (nano)particles on the circumference of the lipid core may require a denser coverage which may be greater than 15%. In some embodiments, therefore, at least 15% of the lipid core circumference is associated or decorated with (nano)particles. At times, at least 15%, 20%, 25%, 30%, 35%, 40%, 45%, 50%, 55%, 60% or more of the circumference is associated with (nano)particles.

The lipid-core comprises a solid lipid or water-insoluble solid polymer that is solid at a temperature between 25 and 100° C. The lipid-core may comprise a single lipid material or a polymer, as indicated, or a combination of lipid materials, or a combination of polymeric materials or a combination of lipid material(s) and polymeric material(s). In some embodiments, the lipid material is a solid triglyceride, a wax fatty ester, solid paraffin, a polymer, or any other solid material capable of solubilizing or dispersing the lipid-soluble or water insoluble agent (active or non-active). In some embodiments, the lipid material constituting the core of a particle of the invention is the active material itself.

Non-limiting examples of lipid materials that can be used as lipid cores and which may or may not carry a lipid-soluble active or non-active material include waxes, beeswax, paraffin wax, hydrogenated vegetable oils, long chain solid fatty alcohols and acids such as cetyl alcohol and stearic acid, Witepsol H32 and others.

In some embodiments, the lipid material is a hard fat with hydroxyl values up to 15. Such hard fats mostly consist of triglycerides with a portion of at most 15% diglycerides and not more than 1% monoglycerides. Examples of such hard fats include the H series of WITEPSOL, e.g., Witepsol H32.

The (nano)particles decorating the circumference of the lipid-core are particulate materials having a size (diameter) typically in the nanometer regime. However, in some instances these (nano)particles may be particles of sizes (diameters) ranging outside of the nanometric regime. In some embodiments, the (nano)particles are of diameters ranging from 20 to 1,000 nm. In some embodiments, these decorating particles are not nanometric and may be of a size up to 10,000 nm (e.g., between 20 nm and 10,000 nm), depending on the size of the lipid core and not extending 10% of the lipid core diameter. For example, when 1, 5 or 10 micron diameter particles are used as decorating the lipid core, particles of at least 10, 50 or 100 microns in diameter, respectively, are obtained. Thus, reference to (nano)particles should be interpreted as referring to the decorating particles which may or may not be nanometric in size (diameter). The term, thus, should be interpreted as particles which are optionally nanometric in size.

The decorating (nano)particles’ size distribution can vary greatly in size.

The decorating (nano)particles comprise or consist of at least one solid material that stabilizes the dispersion, as disclosed hereinabove.

In some embodiments, the decorating (nano)particles are nanoparticles.

In some embodiments, the (nano)particles may be selected of a water-insoluble material that can associate to the surface of the lipid-cores and stabilize the emulsion at the heating stage of preparation. Such materials may be selected amongst metal oxides, water-insoluble metal hydroxides, water-insoluble metal carbonates, water-insoluble metal sulfates, carbon black, silica and clays. Non-limiting examples include zinc oxide, iron oxide, copper oxide, titanium dioxide, aluminum oxide, calcium carbonate, precipitated silica and fumed silica, solid lipid and polymeric (nano)particles, hydroxyapatite or mixtures thereof. In some embodiments, the (nano)particles may be of a material that is functionalized, e.g., amine functionalized. Thus, in some embodiments, the material is any of the aforementioned materials in functionalized form, e.g., silica-NH₂, TiO₂—NH₂, and others.

The functionalization of the decorating (nano)particles can be either by chemical modification, where chemically active agents such as silanes or functionalized silanes, e.g., chlorosilanes, are covalently bound to hydroxyl groups onto silica or other metal oxides to form particles with desired functionalized surfaces that can be cationic, anionic or non-ionic in nature which are suitable to form the particles of this invention. Other surface modification of the decorating (nano)particles may be achieved by adsorbing a functional molecule or polymer, such as amino acids, cationic or anionic polysaccharides such as chitosan, sodium alginate, cholesteric acid, gelatin and proteins. The absorption process can be by immersing the silica, TiO₂ or lipid nanoparticles in a solution of the surface modifying agent for a certain period of time, isolating the particles and drying them. Alternatively, the (nano)particles may be mixed in a solution of the adsorbed molecules and evaporated to dryness. The surface modification of (nano)particles may include surface attachment of ligands or binding sites for molecules or other modifications that may be used for interactions or biding.

The particles of the invention may be used for delivery of active or non-active agents. By appropriately selecting particles of particular sizes and material compositions, various controlled release formulations may be tailored. Generally speaking, formulations of the invention may be provided as slow release, fast release, controlled release, delayed release, release in specific environments, such as environments or media of specific ionic strength, pH, heat, magnetism, light and others.

A unique formulation provided by the invention herein is a controlled release formulation which comprises a population of particles that constantly release its cargo (active or non-active agents) over a period of hours, days and weeks. The cargo may be contained in the lipid-core and/or in the (nano)particles decorating its surface, as disclosed and exemplified herein.

Thus, the invention further provides a formulation comprising a plurality of microparticles, each of said microparticles comprises a lipid-core comprising a lipid material or materials having a melting temperature between 25 and 99° C., wherein the lipid material is either a lipidic active or non-active agent, or at least one material comprising a lipidic active or non-active agent; wherein a total amount of the at least one lipid-soluble or water insoluble agent in each of the microparticles is between 0.1 and 100% w/w; and wherein the lipid-core is surface-decorated with a plurality of (nano)particles permitting stabilization of the microparticles in an aqueous medium.

In some embodiments, the formulation is a controlled or prolonged release formulation exhibiting constant release of the lipidic agent or the at least one lipid-soluble agent into the aqueous medium over a period of at least one hour.

In some embodiments, the microparticle possesses desired properties, including: magnetization, conductivity, super-hydrophobicity, antimicrobial activity, surface adhesion properties, color, physical properties such as smoothness, lubrication, adhesion, metallic nature, interactions with light (absorption or return) and more. These features are embedded onto the surface of the microparticle when specific nanoparticles are used. Several features or functionalities can be embedded onto the surface of a particle so a particle can have a certain color, antimicrobial, superhydrophobic and magnetic, by using a mixture or nanoparticles to make the particle. In the case of a mixture of nanoparticles, the nanoparticles are precoated by absorption or chemical binding of the same surface agent, for example, chitosan or alginate, to allow precise surface composition of nanoparticles.

In some embodiments, the formulation comprises a plurality of microparticles, each of said microparticles comprises a lipid-core comprising (i) a lipid material or materials having a melting temperature between 25 and 99° C., and (ii) at least one lipid-soluble or water insoluble agent (active or non-active); wherein a total amount of the at least one lipid-soluble agent in each of the microparticles is between 0.1 and 100%; and wherein the lipid-core is surface-decorated with a plurality of (nano)particles permitting stabilization of the microparticles in an aqueous medium.

In some embodiments, the formulation comprises a plurality of microparticles, each of said microparticles comprises a lipid-core consisting (i) a lipid material or materials having a melting temperature between 25 and 99° C., and optionally (ii) at least one other agent (active or non-active); and wherein the lipid-core is surface-decorated with a plurality of (nano)particles permitting stabilization of the microparticles in an aqueous medium.

As noted herein, formulations of the invention generally comprise the microparticles, as defined, and an aqueous medium. The agent(s), being active or non-active, may be contained in one or more of (i) the lipid core; (ii) the (nano)particles decorating the circumference of the lipid-core; and/or (iii) the aqueous medium. As such, formulations of the invention may be used as carriers or vehicles for several active or non-active components, allowing use thereof in a variety of fields such as medicine, veterinary, cosmetic, agriculture, in the chemical or bio-industry, food sciences, and others.

In some embodiments, the agent is contained in the lipid-core, wherein the lipid-core comprises an amount of a lipid material solubilizing a lipid-soluble agent. In such embodiments, the agent is a lipid-soluble agent, or presented in a lipid-soluble form. Also, in such cases, the lipid core is free of nanoparticles of any composition and nature, e.g., magnetic nanoparticles, polymeric nanoparticles, metallic nanoparticles, ceramic nanoparticles, semiconductor nanoparticles and others.

In some embodiments, the agent is the lipid-core, hence a lipidic agent, wherein a lipidic carrier or matrix material other than the lipidic agent is not present. In such embodiments, the core may comprise or consist a lipidic agent. In some embodiments, the core consists of a single lipid agent. In other embodiments, the core comprises a main lipidic agent and optionally at least one lipid-soluble agent.

In some embodiments, the agent is contained in the (nano)particles decorating the circumference of the lipid-core. In such embodiments, the agent is a hydrophilic agent or presented in a water-soluble form.

In some embodiments, the agent is contained in the aqueous medium. In such embodiments, the agent is a hydrophilic or water soluble agent or presented in a water-soluble form.

In some embodiments, the size of the (nano)particle can vary from a few nanometers to about 10 micron where the smaller the particle is, the lesser the amount required to form the lipid microparticles and/or the size of the microparticles. In other words, the smaller the particles are, the larger is the coverage surface area and thus fewer (nano)particles are required to form the particles of the invention.

In some embodiments, the surface of the (nano)particles may be modified to improve the ability of the (nano)particles to remain in the interface between the lipid core and the aqueous media. Such a modification can be affected by a chemical modification with activated silane molecules such as 3-aminopropyltriethoxysilane or by surface absorption onto the (nano)particles of alkylamines, fatty acids, amino acids, polysaccharides, polyethylene glycol, acrylic polymers and others. Specific examples include stearyl-amine, propylamine, tetrabutylamine, hexanoic acid, ethanol amine, lysine, phenylalanine, chitosan, sodium alginate, carboxymethyl cellulose, hydroxypropyl cellulose, poly(acrylic-methyl methacrylate) copolymers and hyaluronic acid.

The ability to embed (nano)particles of different properties allows obtaining microparticles of designed and desired properties. (Nano)particles of different properties, sizes and shapes are commercially available from different sources or can be synthesized using procedures published in common academic and technical reports have been used for making microparticles with different surface functionalities. (Nano)particles containing surface polymerizable residues such as vinyl groups, amino and carboxylate groups, isocyanate groups are prepared by chemical bonding to particles surface groups such as hydroxyl group reacted with for example, diisocyanates, acryloyl or methacryloyl chloride, reactive silyl molecules and the like. These surface reactive groups can be polarized by light, heat or by radical polymerization. One application is in 3D printing inks where the printed ink is polymerized by light. Another application is polymerization after coating of a surface to improve adhesion and durability. Moreover, microparticles with metallic surfaces can be sintered or fused by heat or melt and the removal of the inner core by organic extraction, evaporation or degradation.

In some embodiments, the type of (nano)particles should be fitted to the lipid core so that spherical particles are formed. Thus, certain (nano)particles may form desired microparticles with a certain lipid material while other (nano)particles may not form proper microparticles and need to be surface modified in order to form microparticles.

According to the main aspects of the invention, the agent (active or non-active) is contained in the lipid-core and optionally also in or associated with the (nano)particles and/or the medium. The active agents and the non-active agents may vary based on the particular field of use. Generally speaking, particles of the invention or media containing same may be used for pharmaceutical applications, for veterinary uses, as food components and additives, as coloring agents and dyes, for cosmetic applications, as agents for use in agriculture, and in a great variety of other applications. For example, for medical or veterinary uses, the active agents (lipid-soluble or hydrophilic, as may be the case) may be selected amongst pharmaceutically acceptable drugs, wherein non-limiting examples thereof include analgesics, anti-analgesics, anti-inflammatory drugs, antipyretics, antidepressants, antiepileptics, antipsychotic agents, neuroprotective agents, anti-proliferative agents, such as anti-cancer agents, antihistamines, antimigraine drugs, hormones, prostaglandins, antimicrobials (including antibiotics, antifungal agents, antiviral agents, antiparasitic agents), antimuscarinic agents, anxiolytic agents, bacteriostatic agents, immunosuppressant agents, sedatives, hypnotics, antipsychotic agents, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergic agents, electrolytes, gastro intestinal drugs, muscle relaxants, nutritional agents, vitamins, parasympathomimetic agents, stimulants, anorectics and anti-narcoleptics. Non-limiting examples of such materials include oxybenzone, lidocaine, ibuprofen, ketoprofen, escitalopram and others.

In a similar fashion, (non-active) agents used in medicine or veterinary include, in a non-limiting way, vitamins, supplements, natural ingredients, and phytohormones.

For agricultural use, the agent may be a nutrient, a fertilizer, a pesticide, a biocide, plant growth inhibitors, herbicides, hormones and others.

The lipid-soluble agent (either active or non-active) is an agent that is miscible or dispersible in the material of the lipid-core. In other words, the lipid-soluble agent is water-insoluble or a hydrophobic agent or an agent having a LogP value greater than 3. In other cases, the agents have limited solubility in the aqueous media used to make the particles, but can be soluble in a different aqueous media. For example, an agent that is insoluble in a certain pH range, but soluble in other pH range; agents that are insoluble is a certain ionic strength or a mixture of water and a solvent such as ethanol or propylene glycol, but soluble is water of a different ionic strength or a solvent composition. The active agent can be insoluble in the aqueous media and in the lipid core but in a particle size that allows its incorporation as dispersed agent in the lipid core. For example, the agent can be of a size of 1 micron dispersed in a 100 micron particle.

Examples of lipid-soluble agents include pharmaceutical agents, veterinary agents, food components and additives, coloring agents and dyes, cosmetic agents, agents used in agriculture, anti-corrosion agents, anti-crystallization agents, chemical agents, raw materials, bio-materials and agents for other uses. In some embodiments, the lipid-soluble agent is an agent formulated or presented as a lipid-soluble form. In such embodiments, the agent may be, e.g., water soluble, but presented in a form that is lipid-soluble. For example, the agent may be pre-treated with a surfactant or a coating to form a homogeneous dispersion in the solid lipid-core.

Formulations of the invention are typically formed in aqueous media. Such media may contain active ingredients, in addition to those contained in the microparticles or nanoparticles, as disclosed, and may also contain one or more additive such as stabilizers, pH modifiers, pH stabilizers, buffers, detergents, diluents, preservatives, solubilizers, emulsifiers, surfactants, dispersing agents, colorants, antioxidants, oxidizing materials, flavorings, a water miscible organic solvent such as alcohols and esters, salts and others.

In another aspect, the present invention provides a method of preparing a formulation of the invention, the method comprising mixing together (i) a homogenous solution or dispersion of at least one active or non-active agent in a melted lipid core material, and (ii) a dispersion of (nano)particles of a dispersing agent in an aqueous medium; under conditions permitting formation of the formulation.

In some embodiments, the homogenous solution or dispersion is obtained by melting the lipid core material and dissolving (or dispersing) therein the at least one active or non-active agent.

In some embodiments, the dispersion of the decorating (nano)particles is obtained by dispersing the (nano)particles in the aqueous medium (e.g., solution), maintained at a temperature above room temperature (23-30°). In some embodiments, the temperature is above 50° C. In some embodiments, the temperature is 50° C. higher than the melting temperature of the material of the lipid-core.

In other embodiments, the decorating (nano)particles loaded with or associated to active agents are prepared by melt or solvent mixing of the core material and the decorating particles with or without a surfactant so that upon addition to an aqueous solution and vigorously mixing, a uniform dispersion is obtained. In some cases, the mixture may be heated to a temperature that is about 5° C. above the lipid melting point.

In other embodiments, solid lipid nanoparticles (Lipospheres) are prepared from a high melting lipid, where the hot dispersion of the formed lipid emulsion is slowly cooled down to a temperature that is 5° C. below the melting point of the lipid core to form a dispersion. At this stage, another lipid material that melts at a temperature that is about 10° C. below the melting temperature of the lipospheres or below to form an emulsion of the second lipid core decorated with the lipospheres formed in the first stage. The uniform emulsion is cooled down to room temperature or below to solidify the second lipid core to form the solid particles decorated with the lipospheres particles.

In some embodiments, the formulations are added and mixed while hot.

In some embodiments, the hot aqueous dispersion is added into the molten material of the lipid-core and mixed until a uniform dispersion is formed. In some embodiments, the resulting uniform dispersion is further homogenized to reduce the particle size of the formed particles.

In some embodiments, following formation of a uniform dispersion, the dispersion is allowed to cool down. Cooling may be achieved at room temperature or by employing any other means.

Particles according to the invention may be isolated from the dispersion by any means available to the skilled in the art, including for example centrifugation and subsequent isolation or by lyophilization or spray drying. The particles may be isolated as a free-flowing powder and may be thereafter reconstituted by adding an amount of the particles to an aqueous medium.

Also provided are products implementing particles or formulations according to the invention. Such products may be pharmaceutical products, cosmetic products, food products, agricultural products, chemical products, biological products, ink and dye products, flavoring products and others.

Additionally, formulations of the invention may be implemented for use in a variety of unique conditions and applications by varying certain properties of the particles:
-The active agents to be incorporated in either the solid lipid core of the particle or the surface coating particles (by inclusion or association, as disclosed herein) are agents that are insoluble or sparingly soluble in the medium the aqueous formulation is prepared in. In minimizing the solubility of the active agent in the aqueous medium where the formulation is prepared in, one is able to control release of the active agent from the particles. In accordance with the invention, the method of preparation and the aqueous media can be designed so that during preparation, the agent is insoluble in the media. The amount to be solubilized in the aqueous media is dependent on the desired amount of the agent in the particles. For example, an agent that is soluble in acidic media, but not in a basic media may be prepared in a basic solution so that the active agent remains in the particles. Similarly, an agent that is insoluble in a certain ionic strength or a mixture of water and an alcohol (e.g., ethanol), may be used in the media that it is insoluble in.
-The release profile is dependent on the media and the conditions the particles are exposed to. For example, particles that contain active agents that are insoluble in acidic media will not release the agents in the stomach but when passing to the small intestine (having a higher pH). In another example, particles that are loaded with an odor or space disinfectant, should release their content to the air over a period of at least one hour.
-The surface-decorating (nano)particles may be selected to endow the particles with superhydrophobic or superhydrophilic properties or with adhesion properties to certain surfaces such as the lungs or the skin or to certain metal or plastic material.

The particles of this invention can be incorporated into solutions, creams, suspensions, coating materials, 3D printing inks, coating materials, food compositions, pharmaceutical compositions and the like.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C are SEM photos depicting analysis of the Pickering dispersion, at different resolutions. The paraffin wax forms the solid core coated with silica-NH₂ nanoparticles, as shown in FIG. 1C.

FIG. 2 depicts fluorescence microscopy (x10) of solid wax particles decorated with amine modified silica nanoparticles after staining with fluorescamine. The excitation wavelength was set to 365 nm.

FIGS. 3A-C are SEM photos, at different resolutions, depicting analysis of a Pickering dispersion, according to certain embodiments of the invention. The Witepsol® H32 forms the solid core which is coated with silica-NH₂ nanoparticles, as shown in FIG. 3C.

FIGS. 4A-C are SEM photos, at different resolutions, depicting an analysis of the Pickering dispersion, according to certain embodiments of the invention. The hydrogenated vegetable oil forms the solid core which is coated with silica-NH₂ nanoparticles, as shown in FIG. 4C.

FIGS. 5A-C show SEM photos, at different resolutions, depicting analysis of the paraffin particles with 1% ibuprofen, according to certain embodiments of the invention. The paraffin wax forms the solid core coated with silica-NH₂ nanoparticles, as shown in FIG. 5C.

FIGS. 6A-B demonstrate drug release from microparticles in aqueous media, at 25° C., according to certain embodiments of the invention.

FIG. 7 shows a confocal microscopy of hydrogenated vegetable oil Pickering particle with Nile red in the core and fluorescamine at the particle’s surface.

FIG. 8 shows a cross section SEM analysis of the hydrogenated vegetable oil decorated with TiO₂—NH₂. The wax forms the solid core coated with TiO₂-NH₂ nanoparticles.

FIGS. 9A-F present photos of particles prepared from lipid core and chitosan nanoparticles. FIGS. 9E and 9F are the surface and cross-section of a particle.

FIGS. 10A-D show fluorescamine marking of chitosan nanoparticles embedded onto the surface of a lipid microsphere.

FIGS. 11A-D show fluorescence marking of microparticles with two markers to distinguish between the core lipid and surface nanoparticles.

FIG. 12 depicts release of lidocaine from 20% loaded microparticles of Beeswax decorated with chitosan nanoparticles. Release conducted in buffer phosphate 37° C., lidocaine release was determined by HPLC.

FIG. 13 depict release of Ibuprofen from 20% loaded microparticles of Beeswax decorated with starch nanoparticles. Release conducted in water or buffer phosphate, ibuprofen release was determined by UV.

FIG. 14 shows Fluazinam loaded in hydrogenated vegetable oil decorated with modified silica nanoparticles, about 20 microns in diameter, constantly releasing fluazinam when immersed in water at room temperature.

FIG. 15 demonstrates microsphere formation with various ratios of phenylalanine-silica and hydrogenated oil using optical microscopy at X10 resolution.

### DETAILED DESCRIPTION OF EMBODIMENTS

### Example 1: Solid Wax Particles With Surface Colloidal Silica

Materials: Paraffin wax (m.p. 36-38° C.), Silica ~14 nm (Aerosil 200, Lot. 157010913) was obtained from Evonik, fluorescamine was obtained from Sigma Aldrich, acetone (dry), (3-minopropyl)-triethoxysilane (APTES) was obtained from Sigma Aldrich.

### Methods

1. Functionalization of silica: The method was obtained from Yakoov et al. (ACS Omega 2018, 3, 14294-14301). Silica nanoparticles were functionalized by (3-minopropyl)-triethoxysilane (APTES) to introduce amine-functionalized groups onto the particles (silica-NH₂). Silica (1 g) was added to 40 mL of methanol and stirred for complete dispersion. Then, APTES (99% Sigma-Aldrich) was added slowly to the solution for a final concentration of 0.5 M. The reaction was carried out at ambient temperature for 45 min. After silanization, the particles were collected by centrifugation (9000 rpm, 10 min) and rinsed four times with excess methanol. Afterward, the silica-NH2 particles were dried at room temperature under vacuum overnight.

2. Preparation of particles: 0.5 % w/v silica-NH₂ was added to 2 mL deionized water and heated at 90° C. The mixture was homogenized at 22,000 rpm for 30 sec to obtain uniform silica dispersion. 100 µL of melted wax was added and homogenization continued for 30 sec to obtain a uniform O/W emulsion. The formed hot emulsion was cooled down to room temperature by immersing in iced water while continue mixing. The dispersion was stable at room temperature. Particles were isolated by either centrifugation and decanting the water, filtration or lyophilization to form a solid thaw.

3. Instrumentation: Homogenization: POLYTRON homogenizer, Model No. PT 2100 was used for preparing O/W Pickering emulsion. Scanning Electron Microscopy (SEM): They Pickering particles were analyzed using scanning electron microscope Magellan TM 400 L Field Emission Scanning Electron Microscope. Fluorescence microscopy: The microparticles were analyzed in the presence of fluorescamine dye using Olympus FluoView FV10i – Confocal laser scanning microscope.

### Results

1. The formed dispersion can remain as dispersion or the formed particles can be isolated by precipitation and filtration, centrifugation and decantation of the water phase or lyophilized. The dry powder was obtained after air drying which was further characterized using SEM.

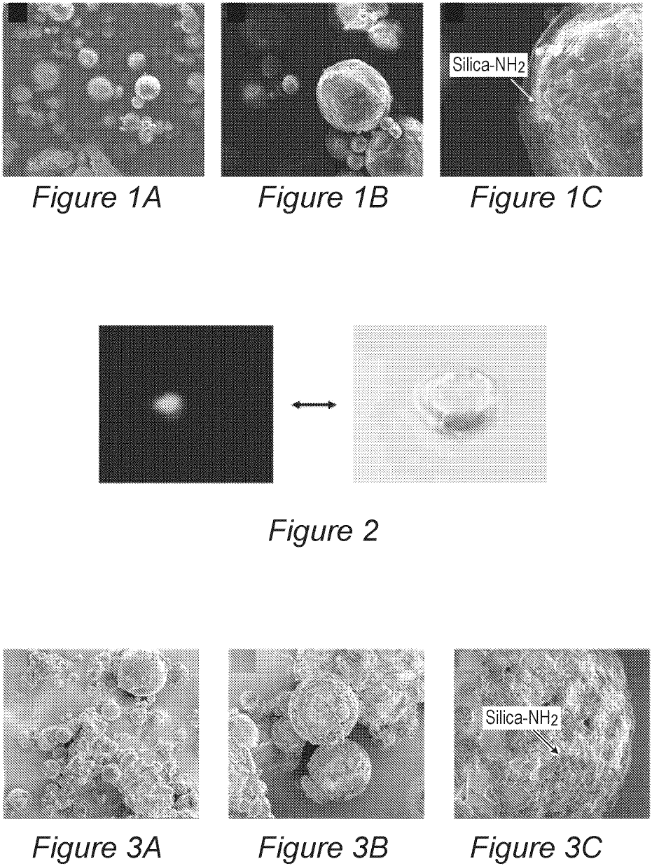

2. The SEM analysis revealed well dispersed particles of size ranging between 30-50 µm. Further, as represented in FIGS. 1, a close view of the particle revealed silica nanoparticle at the surface of the particles.

3. In order to confirm that the wax solid core is supported by silica-NH₂, a fluorescamine dye was used. Fluorescamine interacts with the amines and becomes fluorescent. The dried particles were dispersed in fluorescamine aqueous medium for 10 minutes and analyzed. The excitation wavelength of the dye was set to 365 nm, considering instrument specifications.

The particles glow green in fluorescence, as shown in FIG. 2. This indicates that the silica-NH₂ is at the surface of the wax particles. If the NH₂ groups were not available at the surface, the particles would not have been visible in fluorescence microscopy as confirmed by analyzing particles without the dye.

Uniform, dispersed, and spherical solid particles were prepared using wax and modified silica-NH₂ nanoparticles. The modification of silica using APTES did not disturb the dispersing ability or size of the nanoparticles. SEM analysis revealed the formation of ~50 µm wax particles as solid core with surface embedded silica-NH₂ nanoparticles. The surface silica was further confirmed by fluorescamine binding to silica particles under confocal microscopy.

### Example 2: Preparation of Witepsol® H32 (m.p. 37° C.) and Hydrogenated Vegetable Oil (60-70° C.) Silica Nanoparticles Coated Particles

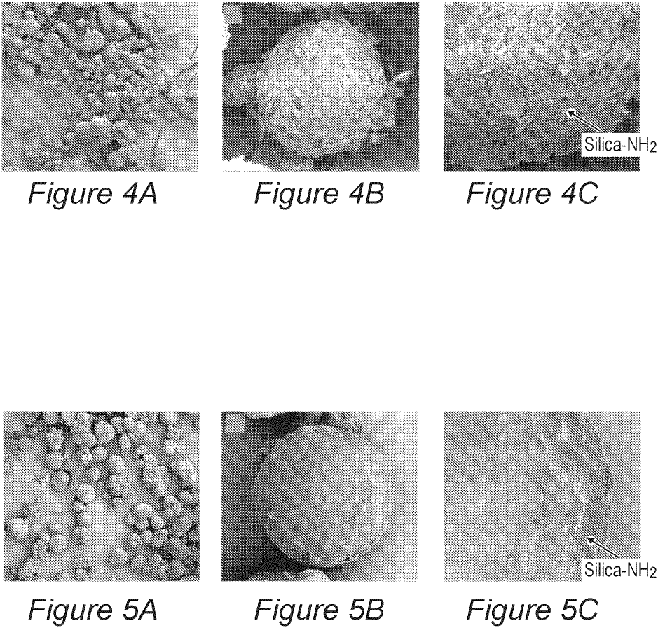

The method of preparation described in Example 1 was used, replacing the wax with Witepsol® H32 as solid core. The SEM analysis revealed well dispersed particles of sizes ranging in between 30-50 µm (FIGS. 3). Similarly, particles with hydrogenated vegetable oil solid cores and surfaces embedded NH₂-silica nanoparticles were prepared. SEM analysis revealed well dispersed particles of size ranging in between 5-40 µm (FIGS. 4).

The developed method was functional and could be applied to varieties of solid lipids and waxes. By adjusting the ratio between the solid lipid and silica-NH₂ size distribution of the particles could be tuned.

### Example 3: Drug Loaded Nano-Shell Solid Particles

Ibuprofen was selected for incorporation in particles. The appropriate amount of drug was solubilized in molten oil at 90° C. and thereafter followed the same procedure as detailed in Example 1.

Paraffin wax particles with 1% Ibuprofen: A dispersion was formed and dried to form a powder that was visualized by SEM. The SEM analysis revealed similar particles to those without the drug, with well dispersed sizes ranging between 30-50 µm (FIGS. 5).

Further preparations: Ibuprofen (1 and 5%), ketoprofen (1 and 5%) and escitalopram (5%) in wax, Witepsol® H 32 and hydrogenated vegetable oil.

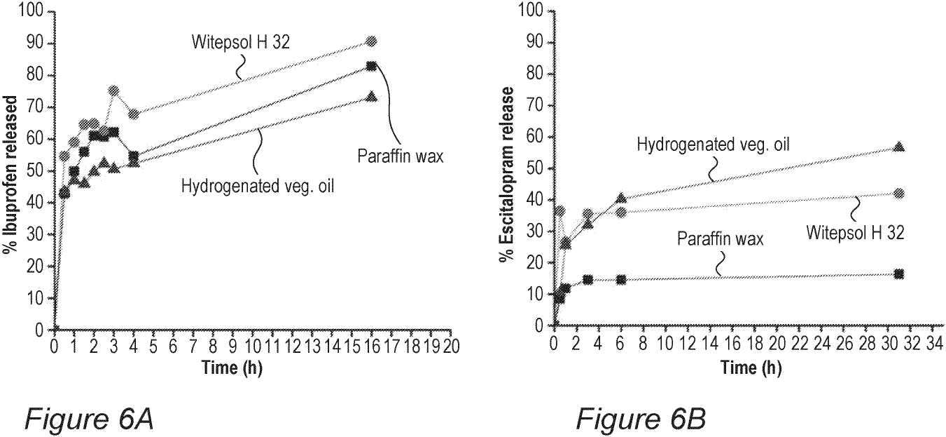

Release of drugs from particles: Standard curve of drug was prepared in 10 mM phosphate buffer at pH 7.2. 10 mg of drug loaded lyophilized Pickering particles were added to 2 mL of buffer and the periodic isolated solution was analyzed by UV. The release was examined at room temperature. This was done in purpose to examine the drug release properties from the Pickering particles. The analysis of initial 30 min of release is considered as the burst release of drugs from the particles (FIGS. 6).

Release of ibuprofen from 1% (w/w) drug loaded Pickering dispersion. The release of ibuprofen was monitored by UV at the wavelength of 223 nm. It was observed that almost all the drug was released within 30 mins, in an aqueous solution (FIG. 6A).

Release of ketoprofen from 1% (w/w) drug loaded Pickering dispersion. Standard curve of ketoprofen was prepared in 10 mM phosphate buffer at pH 7.2 at wavelength of 260 nm. It was observed that almost all the drug was released within 30 mins, in aqueous solution.

The 5% loading of ibuprofen, ketoprofen and escitalopram in the dispersion were similarly prepared. Table 1 summarizes the remaining drug in the particles, after 30 min.

TABLE 1

Encapsulation efficiencies of drugs in particles according to the invention

| Pickering dispersion Solid lipid | % ibuprofen encapsulation (223 nm in UV) | % ketoprofen encapsulation (260 nm in UV) | % escitalopram encapsulation (238 nm in UV) |
|---|---|---|---|
| Paraffin wax | 46 | 53 | 92% |
| Witepsol® H 32 | 57 | 51 | 64% |
| Hydrogenated Veg oil | 56.5 | 57 | 90% |

The particles were checked for the drug release with time. FIGS. 6 provides the release of the drug loaded Pickering particles in a buffer medium. The drug was released in a sustained way. Further, the encapsulation efficiency seemed to be dependent on the compatibility between the drug and the solid lipid.

### Example 4: Characteristics of the Particles in the Presence of the Fluorescence Dyes-Nile Red and Fluorescamine

Nile red, 200 µg was dissolved in the melted wax to prepare the solid core. The dispersion was prepared as described in Example 1. The dispersion was lyophilized to obtain a complete dispersed dry powder.

Fluorescamine, 200 µg/mL of the Fluorescamine aqueous solution was prepared using a stock solution of Fluorescamine in acetone. To 1 mL of Fluorescamine diluted sample, 30 mg of the Nile red containing dispersion was added and kept on shaking for 5 mins and lyophilized. Fluorescamine is water soluble and has the property to bind with primary amines; hence, it interacts only with the silica-NH₂ in the outer-surface of the Pickering particle.

Fluorescence Imaging Analysis: The particles were analyzed under Olympus IX83 inverted microscope. The fluorescent dyes of the same particles were excited separately using default settings in the microscope. Nile red excitation revealed red colored particle and fluorescamine excitation revealed green color particle. The obtained images were then processed using Image J software, that merge both the Nile red and fluorescamine image into one.

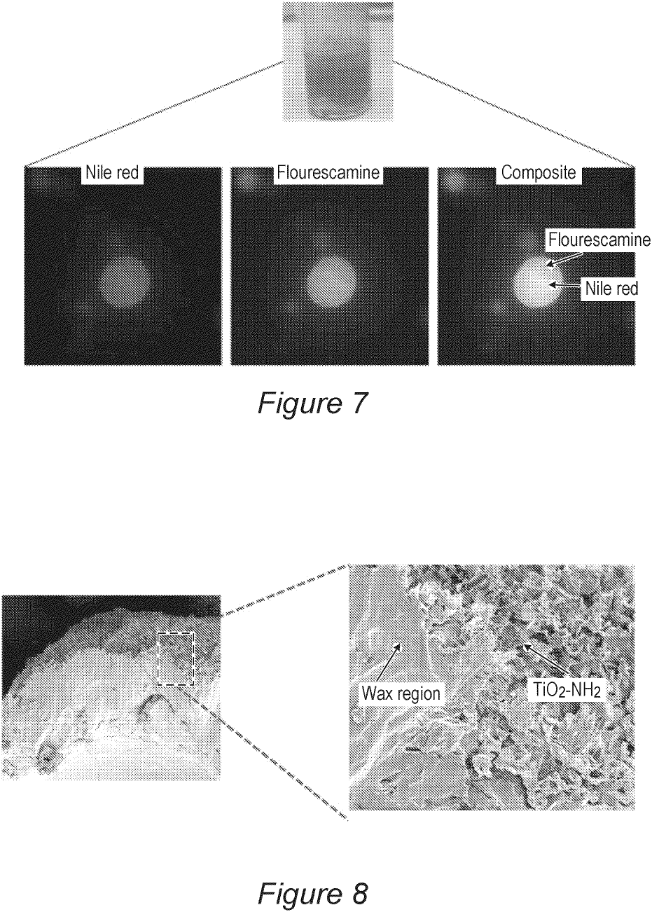

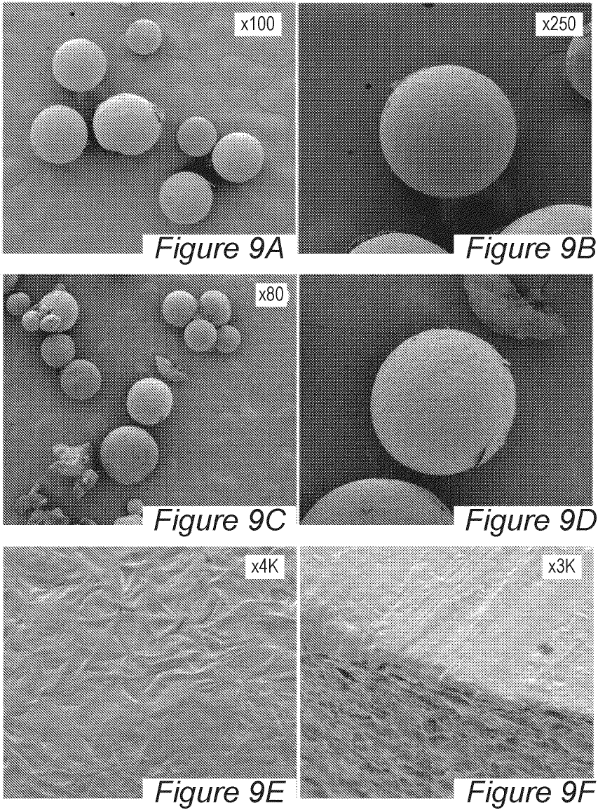

Hydrogenated vegetable oil particles were analyzed under fluorescence microscopy. The particles were dispersed in water and then a drop was added over the glass slides and covered with cover slip. FIG. 7 demonstrates the fluorescence imaging.

FIG. 7 provides information about the selectivity of the dyes over the particles. The black background in the images indicates that there are no free aggregates of the dyes in the aqueous medium. The preference of both dyes can easily be seen in the figure. As such, the Nile red prefers the wax core and the fluorescamine prefers binding to the Silica-NH₂. Being a solid core, it is difficult to differentiate between the red and green fluorescence; however, upon composition, it is seen that the region where there are no Slica-NH₂ the red color is stronger.

The absence of the Nile red dye in the aqueous dispersion indicates the encapsulation efficiencies of the particles to hold hydrophobic molecules. This was observed in FIGS. 6 by observing a sustained release of ibuprofen and escitalopram from the Pickering particles in the release medium.

The distribution of nanoparticles within the core and surface of the microparticles were determined by detecting nanoparticles in the cross-section of particles prepared with silica, chitosan and iron oxide. SEM analysis with EDX did not detect nanoparticles in the cross-section but only on the surface of the particle as shown in FIG. 8.

### Example 5: All Lipid (Nano)Particle Decorating Particles of the Invention

The formation of lipid-based particles where the surface particles lipidic as well as the core, was obtained as follows: In the first step, solid lipid nanoparticles of high melting point lipids such as stearic acid, tristearin or carnauba wax was prepared by either melt method or pre-concentrate method, followed by a second step of the preparation of the microparticle by melt method, using a lipid core that melts at temperatures that are below the melting point of the lipid of the surface. This is demonstrated below:

Step 1: Tristearin was mixed with sterylamine and/or stearol and/or stearic acid and/or phospholipid or a surface-active agent such as Tween and or Span. The mixture was melted at temperature of about 90° C. and mixed well. To the melt, hot water >90° C. containing a minute amount of a surface active agent was added and homogenized to form a homogeneous emulsion. The emulsion was cooled down while homogenizing until a solid dispersion was obtained. The average particle size of this dispersion should be below 2 microns, controlled by the preparation conditions, the ratio of aqueous media to the lipid media, the ratio of surface active agents to lipid and other additives that may be incorporated. These particles contained a lipid active agent that was dissolved or dispersed in the lipid core of this dispersion.

Alternatively, the dispersion could be prepared by dissolving the selected solid lipids in a mixture of surfactants such as Tween, Span, LipoPEG, and phospholipids, Cremophor and a water miscible solvent such as ethanol, propylene glycol, isopropanol, ethyl lactate and N-methyl pyrrolidone (NMP). This homogeneous solution was added to water at room temperature to form a nano-dispersion where the particle size was controlled by the pre-concentrate composition and the properties and amount of aqueous media where the preconcentrate is mixed in.

Step 2: A lipid that melt at a temperature about 10° C. below the melting point of the particles of Step 1 was selected for the core material. Examples are Witepsol, Wax, trilaurin, tricaprin and other lipids that melt at temperature below 50° C. The selected lipid was melted along with the selected active agents to be incorporated in the core. To this melt, the dispersion of Step 1, at an amount of solids that was 10% w/w or less, of the melted lipid, that was diluted in hot aqueous solution and added to the melt while thoroughly mixing or homogenizing to form a homogeneous emulsified dispersion. The mixing continued while the dispersion was cooled down to form the microspheres where the core was the lipid of this step and the surface particles were from Step 1.

The ability to form the particles decorated with lipid particles was dependent on the surface properties of the particles of the first step. The surface should allow interaction with the lipid melt but not incorporation within the melt. The surface properties of the particles of Step 1 can be modified by the addition on lipids with hydrophilic head such as carboxylic acid, amine or PEG chain, for example: fatty acid, fatty amine or fatty alcohol, PEG-fatty acid or a phospholipid of different hydrophilic structure. The size of the final particle can be designed based on the composition of the lipids, the surface active agents, the properties of the aqueous media, the ratio of lipid to particles, and the preparation conditions.

### Example 6: Lipid Microparticles Decorated With Nanoparticles for Controlled Release of Agents

This work utilizes natural materials composed of natural lipid (beeswax) and natural polysaccharides, chitosan and starch, as nanoparticles for the encapsulation of lidocaine (L) and ibuprofen (I) as model drugs. These active agents were chosen as poorly water-soluble drugs with basic (lidocaine) or acidic (ibuprofen) nature. Lidocaine-loaded chitosan-NP-stabilized beeswax microspheres and Ibuprofen-loaded-starch-NP-stabilized beeswax microspheres, prepared by melt dispersion method without use of any surfactant, organic solvents or harsh conditions. These microspheres were fully characterized and the drug release profile was obtained.

### Materials and Methods

White Beeswax was obtained from Nature’s Oil Company, Ohio. Chitosan was obtained from Primex Company. Soluble potato starch was obtained from S.D. Fine-Chem., Mumbai. Lidocaine, Fluorescein isothiocyanate and Fluorescamine were purchased from Sigma Aldrich, Rehovot, Israel. Ibuprofen was obtained from Ethyl Corporation, Orangeburg. Acetic acid and Ethanol were purchased from GADOT Group, Netanya, Israel. Sodium Hydroxide pellets were purchased from J.T. Baker. All the reagents were of analytical grade.

### Preparation of Chitosan Nanoparticles

Chitosan colloidal nanoparticles were successfully synthesized by adjusting the pH value. A typical preparation procedure was as follows: 0.6 g acetic acid was added to 100 mL deionized water to form 0.1 M solution. 0.2 g chitosan was added to 20 mL acetic acid solution and a mixture was continuously stirred for 4 h at room temperature until a clear solution was obtained. The pH value was modified by adding NaOH (1 M) solution, until the transparent chitosan solution was changed to be opalescent (for example, 180 µL of NaOH solution was added for each 2 mL of Chitosan solution). At this moment, the pH of the dispersion was 6.5 with chitosan nanoparticles dispersed in this solution. This nanoparticle dispersion was used immediately for preparation of solid lipid microspheres.

### Preparation of Starch Nanoparticles

Starch nanoparticles were prepared by nanoprecipitation method. Soluble potato starch (2.5 gr) was added to 50 mL of deionized water and the resulting mixture was continually stirred at 90° C. for 1 h. The heating was turned off and 50 mL of Ethanol was added dropwise using additional funnel. During an addition of Ethanol white precipitate started to form. After the dispersion was reached room temperature another portion of 50 mL ethanol was added dropwise. The resulting mixture was centrifuged at 9000 rpm for 5 min. After supernatants were removed 20 mL ethanol was added and the mixture was centrifuged. These washing cycles with ethanol performed two more times in order to remove the water. The remaining white solid was dried under reduced pressure.

### Preparation of Lidocaine-Loaded Solid Lipid Microspheres (SLM) Stabilized With Chitosan NP

Lidocaine loaded particles were prepared using a melting dispersion method. Beeswax was heated and melted in oil-bath at 90° C. and solid Lidocaine (base form) added whilst stirring to form a beeswax-lidocaine clear mixture (Lidocaine 10% w/w). Separately, Chitosan-NP dispersion in water was heated in the same oil-bath with stirring at 1000 rpm. The melted mixture of Lidocaine in Beeswax was subsequently added in one portion to the Chitosan dispersion with constant stirring and stirring was continued for 3 min more (70 µL of melted mixture were added to each 2 mL of 1% Chitosan dispersion). The resultant mixture was cooling in a water bath with stirring till reached the room temperature. During the cooling microspheres were formed. Blank-SLM were prepared using the same method but without an addition of the drug.

### Preparation of Ibuprofen-Loaded Solid Lipid Microspheres (SLM) Stabilized With Starch NP

Ibuprofen loaded particles were prepared using the same method. Beeswax was melted in oil-bath at 90° C. and solid Ibuprofen added whilst stirring to form a beeswax-ibuprofen clear mixture (Ibuprofen 22% w/w). Separately, starch-NPs were accurately weighted and the deionized water was added. The resulting mixture was sonicated for 5 min in order to make uniform dispersion. This dispersion was heated in the same oil-bath with stirring at 1000 rpm for 5 min. The melted mixture of Ibuprofen in Beeswax was subsequently added in one portion to the Starch-NPs dispersion and stirring was continued for 8 min more (70 µL of melted mixture were added to each 4 mL of Starch dispersion containing 80 mg of dry Starch-NP). The resultant mixture was cooled in an ice-water bath with stirring. During the cooling microspheres were formed. Blank particles were prepared using the same method but without an addition of the drug.

### Particles Characterization

### Mean Particle Size Measurement

The average particle size of chitosan and starch nanoparticles was determined by using ZetaSizer NANO (Malvern Instruments, UK). Chitosan NP and Starch NP dispersions were diluted with deionized water and were equilibrated to room temperature for 2 min. A 1 cm path length Brand® disposable UV-transparent cuvettes were used for the particle size measurement. All measurements were performed at a constant temperature of 25° C.

### Scanning Electron Microscopy

SEM photographs were taken using a scanning electron microscope FEI Quanta 200 SEM (Brno, Czech Republic) at suitable magnification at room temperature. The photographs were observed for morphological and structural characteristics and to confirm the spherical nature of microparticles. The samples were mounted on aluminum stubs, using double sided adhesive tape and Pd/Au coated under vacuum for 60 s using a sputter coater (SC7620, Spatter coater, UK). The average particle size of the microspheres was noted from the SEM studies.

### Transmission Electron Microscopy

The morphological observations of microparticles were obtained by using JEM-1400 PLUS, Japan, the 120 kV model in JEOL’s new PC controlled TEM range. A drop of NP dispersion was placed onto a carbon-coated copper grid followed by the removal of excess dispersion using a filter paper. It was then negatively stained using NanoVan (Nanoprobes, USA) or Uranyl acetate (2%) and air-dried at room temperature for 30 min. The grid was then ready to be examined under TEM.

### Fluorescent Microscopy

Fluorescent microscopy imaging was performed on Nikon Spinning Disk Microscope. The system includes the Yokogawa W1 Spinning Disk with wide field of view. Two discs are integrated with 25 µm and 50 µm pinholes, for optimal imaging with different objectives. The system is mounted on the new Nikon motorized fluorescent microscope Ti2E and equipped with 405/488/561/638 nm lasers. The system includes the NIS Elements software package for multi-dimensional experiments, with JOBS for high content acquisition and analysis, deconvolution, tracking, 3D automatic measurements.

### Preparation of Fluorescent Solid Lipid Microspheres for Imaging

Dried Beeswax-Chitosan and Beeswax-Starch microspheres (10-20 mg) were added to 2 mL of ddH₂O, followed by addition of 50 µl of Fluorescein isothiocyanate/Fluorescamine solution (1 mg/mL, in DMSO). The mixture was shaken for 1 h in the dark at room temperature. The liquid was removed and the remaining particles were washed 5 times with water in order to remove all unreacted dye. The obtained microspheres were imaged using Nikon Spinning Disk Microscope.

### Assay of Lidocaine and Ibuprofen by UV

The amounts of Lidocaine and Ibuprofen were measured by UV absorbance using an ultraviolet spectrophotometer (UV-Visible Spectrophotometer, Ultrospec 2100 pro, Amersham Biosciences). Lidocaine was measured at a wavelength of 215 nm in 10 mM PBS, and a wavelength of 242 nm in chloroform. The standard curves were constructed in a concentration range of 5-25 µg/mL in 10 mM PBS with R² value of 0.99 and in a concentration range of 7-100 µg/mL in chloroform with R² value of 0.995. Ibuprofen was measured at a wavelength of 223 nm in 10 mM PBS or water and a wavelength of 264 nm in chloroform. The standard curves were constructed in a concentration range of 3-70 µg/mL in 10 mM PBS with R² value of 0.9993 and in a concentration range of 15-800 µg/mL in chloroform with R² value of 0.9935.

### Determination of Encapsulation Efficiency and Loading Capacity

Encapsulation efficiency is percentage of drug encapsulated in the microspheres relating to initial quantity used. 10 mg of microspheres were dissolved in 4 mL of chloroform. The absorbance of the drugs was measured using a UV-VIS spectrophotometer after appropriate dilution in order to reach calibration curve concentrations. The measured absorbance was then converted to the amount of drug by using standard calibration curve.

### In Vitro Drug Release

In vitro release of Lidocaine and Ibuprofen from microspheres was studied using 10 mM PBS or ddH₂O as dissolution medium at 37° C., 150 rpm (using shaking incubator). 10 mg of microspheres were mixed with 5 mL of H₂O or buffer and placed in the incubator. Three samples of each formulation were run at same conditions. A 3 mL samples were withdrawn at predetermined time intervals and an equal volume of fresh dissolution medium was replaced. Collected samples were suitably diluted and then analyzed for drugs contents by measuring the absorbance at 215 nm for Lidocaine samples in 10 mM PBS and at 223 nm for Ibuprofen samples. The concentration of lidocaine and ibuprofen in test samples was calculated using calibration curves for each drug in H₂O or 10 mM PBS.

### Results and Discussion

### Lidocaine Loaded Beeswax-Chitosan Microspheres

The transmittance of the aqueous chitosan solutions decreased with increasing pH values. At high pH (near 6.6, pKa of chitosan), chitosan’s amines becoming deprotonated and chitosan could undergo interpolymer associations that lead to nanoparticle formation. TEM images of these nanoparticles revealed spherical particles with diameter rage between 200 and 800 nm.

This nanoparticles dispersion was used as stabilizing material for the formation of solid lipid microspheres. Several parameters were studied in order to achieve spherical form with maximum yield. These p arameters included: amount of wax added to aqueous dispersion of chitosan NP, amount of base (NaOH) added that influence the pH which by itself influence the concentration of precipitated chitosan NP, temperature of oil bath where all ingredients were mixed, the stirring speed and the stirring time. The right ratio between the amount of beeswax and the concentration of chitosan NP enables the maximum yield of particles formed without any residual of unreacted wax. For comparison, without the addition of chitosan nanoparticles, the process failed and resulted in formation of aggregate cake of solid wax. It may be due to the repulsion resulting from high interfacial tension between the hydrophobic waxy material and external aqueous phase.

The formed spherical microparticles were collected and dried in an open air. The same procedure was used for the preparation of lidocaine-loaded microspheres. For this purpose, Lidocaine (mp 68.5° C.) was mixed with beeswax at 90° C., temperature which allows both ingredients to appear at their melted form, and thus, to accelerate mixing. This melted mixture was added to the chitosan nanoparticles dispersion in the same matter as pure wax was added. Important to notice, that the amount of Lidocaine drug mixed with wax has huge influence on yield of microspheres formed. Thus, while the percentage of the Lidocaine increases, the yield of formed particles decreases. For this reason, the melted mixture contained 10% of Lidocaine which allowed the reasonable formation yield. The morphological structure of the synthesized microparticles with or without Lidocaine drug was examined using SEM. It was found that there was no visible difference in shape, size and their surface morphology. SEM photographs showed that the wax microspheres were spherical in nature and had a smooth surface with size distribution of 300-600 µm.

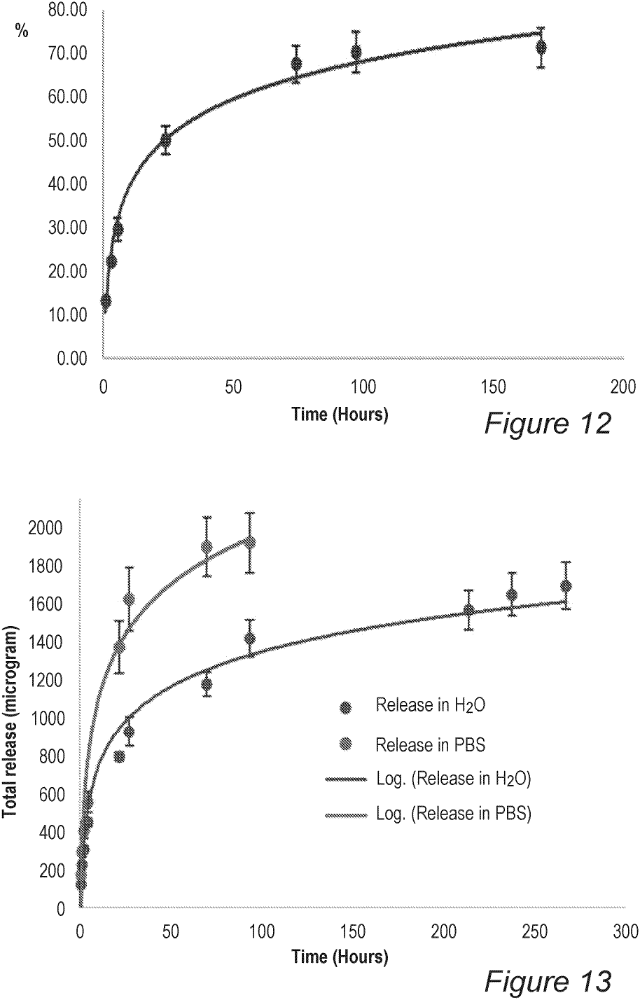

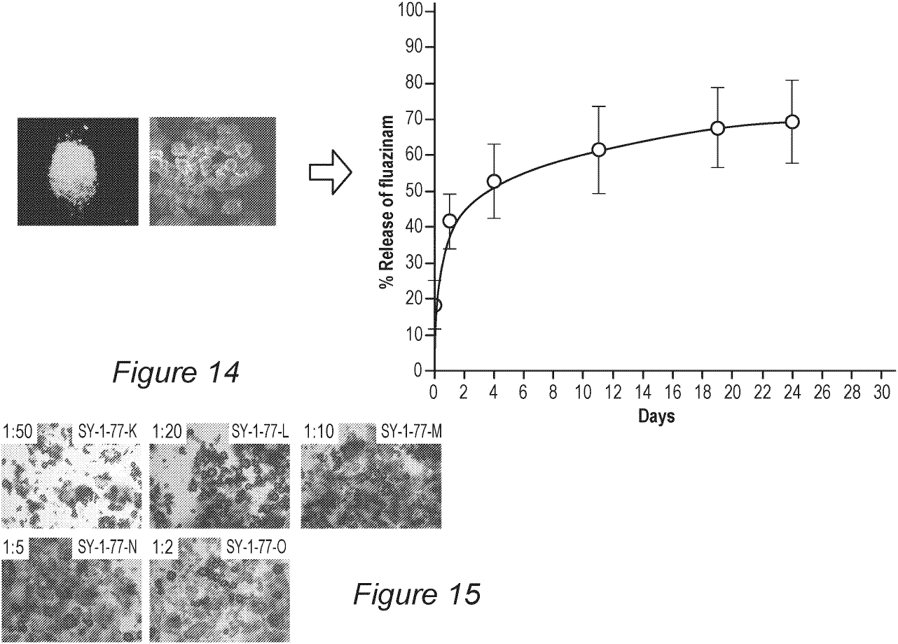

The enlarged images revealed unique “wave”-shaped surface. One of the spherical particles was successfully cut in order to understand difference between the inner and outer phases. The SEM images indicated that the core has more homogenic nature while the outer phase is very porous with rough texture which is characteristic to polysaccharides. The in vitro release of Lidocaine from particles is given in FIG. 12.

### Ibuprofen Loaded Beeswax-Starch Microspheres

Commercially available soluble potato starch consists of smooth oval microparticles. In order to serve as a stabilizing material in the lipid microspheres preparations there is a need for starch nanoparticles. These starch nanoparticles were prepared by nanoprecipitation method from commercial microparticles. The formed particles were spherical in shape and had an average diameter of 306 nm, according to DLS measurements.

These nanospheres were used in further lipid microspheres preparation. Mixture of starch NP in water was sonicated prior to use in order to get homogeneous dispersion. Several factors that can influence the particles formation have been studied -amount of starch NP, amount of beeswax, stirring rate, temperature and the stirring time. It was recognized that the last parameter has of greatest importance on particle yield. Thus, only when enough time was given for the mixing of wax with starch (10 min instead of usual 3 min), nice spherical particles were formed upon cooling. The same procedure was used for the preparation of ibuprofen-loaded microspheres. For this purpose, Ibuprofen (mp 75-78° C.) was mixed with beeswax at 90° C., temperature which allows both ingredients to appear at their melted form, and thus, to accelerate mixing. In this case, it was possible to prepare rather concentrated mixture which contained 20% of Ibuprofen and yet to get good yield of microparticles without any residue wax. These microspheres were characterized by SEM imaging. SEM photographs showed that the wax microspheres were spherical in nature and had a smooth surface with size distribution of 300-600 µm.

### Fluorescent Labeling of Solid Lipid Microspheres

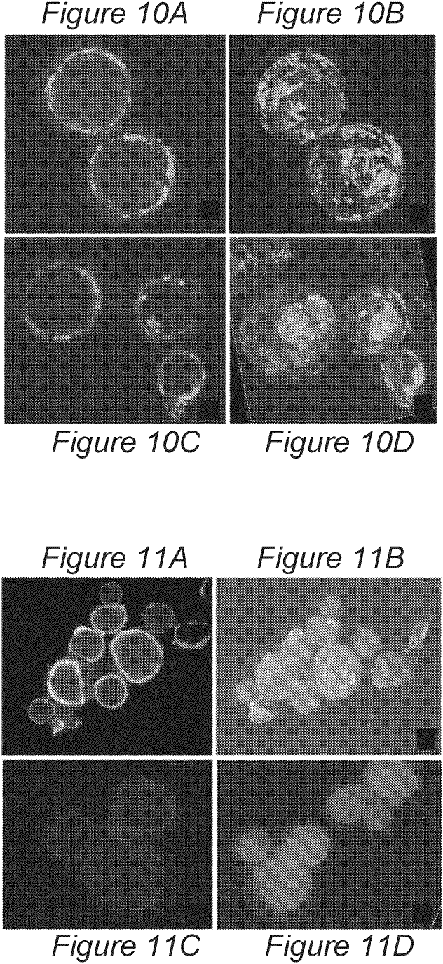

The fluorescent labeling methods for polysaccharide labeling have been widely researched in recent years. Fluorescent marker, fluorescein isothiocyanate (FITC), was conjugated to the chitosan molecule and was used to study chitosan-mucin interactions and the biodegradation and distribution of chitosan in mice. Through covalent bonding, the primary amino group of chitosan combines with isothiocyanate group (N═C═S) of fluorescein isothiocyanate to label the chitosan. The chitosan labeled by fluorescein isothiocyanate has a high fluorescence intensity, light stability and solid combination. In this work this fluorescent labeling compound was used in order to visualize the polysaccharide coatings of prepared Solid Lipid Microspheres. Thus, the microparticles were allowed to mix with the dye solution, intensively washed with water to remove the unreacted dye and visualized immediately by fluorescent microscope (FIGS. 10)

In order to distinguish between the wax core and the polysaccharide coatings the microspheres were fluorescently labeled using two different fluorescent dyes. The Nile red dye was incorporated in Beeswax prior to the formation of microspheres to mark the core. After these particles have been formed using ChitosanNP as stabilizers, they were colored with FITC dye. When the particles treated with FITC were excited at two wavelengths (561 nm and 488 nm) fluorescent images revealed red core and green coatings (FIGS. 11A-B). In order to confirm the presence of chitosan coating, the obtained microspheres were treated with different dye, Fluorescamine. Fluorescamine is a coloring reagent developed by Weigele et al. for amino acid analysis. This molecule is not fluorescent by itself but upon reaction with primary amines forms a fluorescent product. Thus, if the nanoparticles of chitosan are present on the surface of beeswax, the Fluorescamine should react with NH₂ group, present in Chitosan, forming a fluorescent product, which has an excitation maximum at 390 nm. These particles were imaged using 405 nm laser to visualize the surface coating labeled with Fluorescamine (blue, FIG. 11C) and using two lasers (405 nm and 561 nm) to visualize the wax core and chitosan coating (red and blue, FIG. 11D).

### Drug Encapsulation and Loading Capacity of Solid Lipid Microspheres

The loading capacity is usually expressed as the ratio of the weight of entrapped drug and the total weight of the lipid and coating particles in percentage. Some of the factors which affect the loading capacity of a drug in a lipid are the solubility of the drug in the lipid melt, the miscibility of the drug and the lipid melt, the chemical and physical properties of the drug. Thus, if the melting point of drug is similar to the lipid melting point, it is possible to melt them together, which highly increase the loading capacity. In this work we chose to use drugs which have similar or close melting point as beeswax. Thus, under the procedure conditions, 90° C., both wax and drug materials are melting (beeswax melting point is 62-64° C., Lidocaine melting point is 68° C., Ibuprofen melting point is 75-77° C.), allowing good mixing. The loading capacity of Lidocaine in Beeswax-Chitosan MSs was found to be 4.9 % and of Ibuprofen in Beeswax-Starch MSs was found to be 15.7% (average of three independent experiments for each formulation).

### Drug Entrapment Efficiency

Drug entrapment efficiency is calculated in order to determine the amount of drug that is actually retained in the microparticles related to the amount of drug that was mixed with lipid before particles formation, and usually expressed as a percentage. The chemical nature of drug, like water solubility or existence of basic or acidic groups in molecule can influence the entrapment efficacy. Thus, in the case of solid lipid particles formation using chitosan nanoparticles, the pH of the dispersion is 6.5, therefore, drugs containing acidic group, like Ibuprofen, will become deprotonated, leading to their salt formation and increased water solubility. This way some portion of the drug will diffuse from lipid to the aqueous medium during particles preparation, leading to decreasing in entrapment efficiency. In addition, as a water solubility of drug is increasing, the efficacy of encapsulation using hot melting method is decreasing. Therefore, Ibuprofen-loaded microspheres obtained from beeswax and starch have been formed with good entrapment efficiency (71%), as Ibuprofen is only very slightly soluble in water (21 mg/L, at 25° C.) and the drug remains in its neutral form during particle formation. Lidocaine molecule contains a basic group (tertiary amine) which remains neutral during the particles formation using Beeswax-Chitosan (pH 6.5) but has higher water solubility (410 mg/L at 30° C.) leading to lower entrapment efficacy (49%).

### Studies of Drugs Release From Solid Lipid Microspheres

In vitro drug release for the obtained microspheres was studied in 5 ml of 10 mM buffer phosphate solution, pH 7.4 or in ddH₂O at 37° C. The release profile of Lidocaine from Beeswax-Chitosan

The release profile was fitted logarithmic function, with initial burst release at first 24 h, where about 70% of drug was released and the remaining amount of drug was released during at least one week. FIG. 13 illustrates the average of three different experiments.

The release profile of Ibuprofen from Beeswax-Starch MSs was studied at two dissolution media, buffer PBS and ddH₂O at 37° C. As Ibuprofen possesses the acidic group — COOH, the pH of the solution has a very meaningful influence on its release rate. Thus, this statement can be proven by observing two release profiles depicted in FIG. 13. Both graphs followed the logarithmic behavior, with boost release of the drug during the first day. But it can be recognized that in case of the particles’ dissolution in PBS pH 7.4, most of the drug was released during 4 days, while in case of using water instead of buffer, the release was continued for 7 days more. Thus, by changing the dissolution medium (or formulation) it is possible to control the rate of drug release from these microparticles, leading to the desired one, depending on application.

Summary: Natural particle-stabilized solid-lipid microspheres were prepared by melt dispersion technique. Using a natural lipid melt and nanoparticles from natural source spherical microparticles were produced. These microspheres were prepared also using drug-lipid melt for the production of drug-loaded microparticles. Beeswax, chitosan and starch, due to their lower toxicity, absence of solvents, surfactant or emulsifiers in the production process can be suitable for coating of drugs which need to be released in controlled rate.

### Example 7: Oxybenzone-Loaded Solid Lipid Microparticles Stabilized by TiO₂ Nano-Shell

Microparticles were prepared using hot melt homogenization technique from three different types of solid lipids, soy wax, cetyl alcohol and trilaurin, approved for cosmetic applications, using TiO₂ nanoparticles (nano-shell) as stabilizing material.

In this method no surfactant was applied, but instead, nanoparticles of TiO₂ were used as stabilization material, leading to the formation of solid spherical particles of micrometer size (10-50 microns). Microparticles were obtained using the three lipids: Soy wax, Cetyl alcohol and Trilaurin loaded with four different Oxybenzone concentrations: 20%, 25% 33.3% and 50%. The total yield is >90%, the isolated powder yield was in the range of 70-85% due to loss of materials during isolation of particle. The loadings ware similar to the entry loading and the average particle size of all particles was in the range of 23 to 35 microns.

Cream formulations containing 10% were prepared and sent for SPF (Sunscreen Protection Factor) evaluation. Cream was prepared using Water, Cocoa Butter, Soy lecithin, HPMC, PG and Propyl Paraben.

Earlier, another Oxybenzone-loaded microparticles were prepared as well, using Trimyristin and modified TiO₂, were characterized as well and their SPF values were measured. The SPF for the free oxybenzone in cream or the titanium oxide-lipid microparticles without oxybenzone was in the range of 2.9-3.4 while all cream samples containing particles loaded with oxybenzone and titanium oxide nano-shell, demonstrated SPF in the range of 7-8.5.

### Example 8: Lipid Particles Decorated With Metal Oxides

Microparticles were prepared using Titanium dioxide (TiO₂). Microparticles of paraffin wax and Hydrogenated Vegetable Oil were prepared and examined. A fine free flowing dry powder was obtained after lyophilization which was further characterized using SEM. The SEM analysis revealed well dispersed particles of size ranging in between 10-50 µm.

Drug encapsulation in wax microparticles: The encapsulation and release of 5% of ibuprofen, ketoprofen and escitalopram was studies. The encapsulation efficiency of the drugs ranged from 50 to 95%.

The cross section of the TiO₂—NH₂ onto hydrogenated vegetable oil particles was examined using SEM. This analysis proved that the decorating TiO₂ nanoparticle remains only at the surface and nothing in the core.

Particles decorated with Iron oxide: Microparticles were prepared using iron oxide nanoparticles. The surface of the iron oxide was modified using silane. The silane modified metal oxide is abbreviated as Fe₂O₃—NH₂. The preparation method is same as described above. Microparticles with paraffin wax and hydrogenated vegetable oil decorated with iron oxide were obtained as fine dry powder after lyophilization which was further characterized using SEM. The SEM analysis revealed well dispersed particles of size ranging in between 10-50 µm.

### Example 9: Magnetic Microparticles

Magnetic nanoparticles of different sizes, shapes and compositions are commercially available or synthesized in large scale iron oxide or iron metal nanoparticles.
Antimicrobial microparticles
Colored Microparticles
Conductive Microparticles

### Example 10: Formulation of Microparticles in Carriers

The microparticles of this invention were granulated into large granules by mixing the particles with poly(vinyl pyrrolidone) or polyvinylalcohol aqueous solution which after water evaporation large granules were obtained. The size of the granules can be tailored by the process or by grinding the large granules. The microparticles may be a mixture of microparticles with different active agents or/and, mixed with inert particulate solids such as clay, microcrystalline cellulose, starch and the like, prior to granulation. In a typical preparation,

Drug loading: DEET insect repellent 30% (w/w): 30% w/w of DEET was loaded into the microparticles. The selection of the 30% drug content was based on the dissolving the DEET into the molten oil and then cooled down. The DEET loading was examined for 1:1, 1:2, 1:10 and 1:20 ratio SiO₂-NH_{2:}hydrogenated vegetable oil. The DEET loaded particles were tested by weight loss in the DEET loaded 1:10 ratio was examined. Accelerated temperature of 50° C. was used. The weight sample was heated to 500° C. and monitored the weight loss for 1 h. 2% decrease in the weight loss of the DEET loaded microparticles was observed.

Fluazinam 30% (w/w): 30% w/w of fluazinam was loaded into particles. The selection of the 30% drug content was based on the dissolving the drug into the molten oil and then cooled down. The drug loading was examined for 1:1, 1:2, 1:10 and 1:20 ratio SiO₂—NH₂—hydrogenated vegetable oil by taking ~10 mg of the drug loaded particle material and vortexing strong for 15 minutes in 5% Tween 20 aqueous solution. Fluazinam release is shown in FIG. 13.

### Example 11: Modification of Silica Nanoparticles by Physical Adsorption and Identifying the Formation of Solid Lipid Microparticle

The aim was to investigate the solid lipid formation using physically modified nanoparticles (NP). Surface modification of NP should be done by absorption of cationic or anionic polysaccharides, amino acids, fat, PEG, etc. Silica nanoparticles was used and surface modified by physical adsorption of materials such as, chitosan, alginate, cholesterol and amino acid.

Materials: Silica ~14 nm (Aerosil 200, Lot. 157010913) was obtained from Evonik; Cholesterol; low mol. wt. Chitosan; medium viscosity sodium alginate; DL-phenylalanine.

Choice of material: The coating material was chosen such that they are practically insoluble in aqueous conditions. This will diminish the chances of the coating that could erase from the silica nanoparticles while pickering preparation.

### Method

Surface modification of the Silica NPs: 5% w/w coating agent was used in the total material unless specifically stated.

Cholesterol coating over NPs: 50 mg of cholesterol was dissolved in 50 mL ethanol. Thereafter, 950 mg of the silica NP was dispersed by vortex mixing for 30 mins. The mixture was then vacuum dried using rotaevaporator.

DL phenylalanine coating over NPs: 50 mg of phenylalanine was dissolved in 50 mL ethanol. Thereafter, 950 mg of the silica NP was dispersed by vortex mixing for 30 mins. The mixture was then vacuum dried using rotaevaporator.

Chitosan coating over NPs: 50 mg of low mol wt. chitosan was dissolved in 50 mL acidic water at pH 4. Thereafter, 950 mg of the silica NP was dispersed by vortex mixing for 30 mins. The mixture was then freeze dried using a lyophilizer.

Sodium alginate coating over NPs: 25 mg of medium viscosity sodium salt of alginic acid was dissolved in 50 mL water. 25 mg of the coating material was used because of the high viscosity of the sodium alginate. Thereafter, 975 mg of the silica NP was dispersed by vortex mixing for 30 mins. The mixture was then freeze dried using a lyophilizer.

### Preparation of Microparticle Dispersion

Step 1. Various w/w ratios of surface modified silica were added to 2 mL water and heated at 90° C. The mixture was homogenized at 22,000 rpm for 35 sec to obtain a uniform NP dispersion.

Step 2. In a separate vial, solid lipid was heated at 90° C. 100 µL of melted wax was added to 2 mL of hot dispersion and homogenized at 22,000 rpm for 35 sec to obtain a uniform O/w emulsion.

Step 3. The O/w emulsion from Step 2 is cooled down at room temperature and then air dried for further analysis.

Results: The obtained surface modified silica NPs are powdered material, easily dispersible in water upon vortexing or under shear force such as under homogenizer.

CHOLESTEROL-SILICA + PARAFFIN WAX: Various ratios of NP to Lipid were selected. No microparticles were formed

CHOLESTEROL-SILICA + CETYL ALCOHOL: Various ratios of NP to Lipid were selected. Good microparticles were obtained for formulations prepared from cholesterol-silica and cetyl alcohol in the ratio, 1:50, 1:20 and 1:10.

CHOLESTEROL-SILICA + Hydrogenated Vegetable oil: Various ratios of NP to Lipid were selected. Good microparticles formation is observed between cholesterol-silica and hydrogenated vegetable oil in the ratio, 1:50, 1:20; 1:10 and 1:5.

PHENYLALANINE-SILICA + PARAFFIN WAX: Various ratios of NP to Lipid were selected. Good microparticle formation was observed between phenylalanine-silica and paraffin wax in the ratio, 1:50.

PHENYLALANINE-SILICA + CETYLALCOHOL: Various ratios of NP to Lipid were selected. Good microparticle formation is observed between phenylalanine-silica and cetyl alcohol in the ratio, 1:50, 1:20, 1:10 and 1:5.

PHENYLALANINE-SILICA + Hydrogenated Vegetable oil: Various ratios of NP to Lipid were selected. Good microparticles formation was observed between phenylalanine-silica and h veg oil in the ratio, 1:50, 1:20, and 1:10.

TABLE 2

Non-limiting examples of particles prepared according to the invention. ++ successful microparticles formation; + good microparticles formation; – no formulation obtained

| | | | | | | |
|---|---|---|---|---|---|---|
| Nanoparticle (NP) | Lipid (L) | Nanoparticles: Lipid ratio | | | | |
| | | 1:50 | 1:20 | 1:10 | 1:5 | 1:2 |
| Cetyl alcohol | Silica | ++ | ++ | ++ | ++ | + |
| Cetyl alcohol | Cholesterol-Silica | ++ | + | + | - | - |
| Cetyl alcohol | Phenylalanine-Silica | ++ | ++ | ++ | + | + |
| Paraffin wax | Silica | - | - | - | - | - |
| Paraffin wax | Cholesterol-Silica | - | - | - | - | - |
| Paraffin wax | Phenylalanine-Silica | + | - | - | - | - |
| H. Veg oil | Silica | - | - | - | - | - |
| H. Veg oil | Cholesterol-Silica | ++ | ++ | ++ | - | - |
| H. Veg oil | Phenylalanine-Silica | ++ | ++ | ++ | - | - |

This study indicates that surface coating of nanoparticle improve their ability to form nano-shell microparticles.

### Example 12: Nano-Shell Coated Microparticles From Different Nanoparticles and Lipid Inner Core. Effect of Composition

Aim of the proposed research: Literature reveal the Pickering emulsion with oils and solid particles. It means, in either case, Oil in water or Water in oil emulsions, the inner core is liquid supported by a surface layer of solid particle.

Herein, we propose to use a solid wax, fats or lipids with combination with colloidal silica or solid particles to create Pickering dispersions with a solid core and with controlled dimensions. The solid lipid particles find wide application is food industry, cosmetics, and medical fields.

Method: Various amount of nanoparticle (2, 5, 10, 20 50 mg) was added to 2 mL water and heated at 90° C. The mixture was homogenized at 22,000 rpm for 35 sec to obtain a uniform dispersion. In a separate vial, the solid lipid was heated at 90° C. 100 µL of melted wax was added to 2 mL of hot dispersion and homogenized at 22,000 rpm for 35 sec to obtain a uniform O/w emulsion. The O/w emulsion is cooled down at room temperature and then dried for further analysis. The ratios of NP to lipids is considered as, 1:50, 1:20. 1:10, 1:5 and 1:2.

### Instrumentation

Homogenization: POLYTRON homogenizer, Model No. PT 2100 was used for preparing O/w Pickering emulsion.

Scanning Electron Microscopy (SEM): They Pickering particles were analyzed using scanning electron microscope Magellan TM 400 L Field Emission Scanning Electron Microscope.

Chemical modification of silica-nanoparticle surface: The method was obtained from Yakoov et al. (ACS Omega 2018, 3, 14294-14301). Silica nanoparticles were functionalized by (3-minopropyl)-triethoxysilane (APTES) to introduce amine-functionalized groups onto the particles (silica-NH2). Silica (1 g) was added to 40 mL of methanol and stirred for complete dispersion. Then, APTES (99% Sigma-Aldrich) was added slowly to the solution for a final concentration of 0.5 M. The reaction was carried out at ambient temperature for 45 min. The particles were collected by centrifugation (9000 rpm, 10 min) and rinsed four times with excess methanol. Afterward, the silica-NH₂ particles were dried at room temperature under vacuum overnight.

TABLE 3

List of lipids tested

| Lipids | Melting point (°C), literature |
|---|---|
| PARAFFIN WAX | 47 - 65 |
| CETYL ALCOHOL | 49 |
| STEARIC ACID | 69-72 |
| DYNASAN® 114 | 55-60 |
| DYNASAN® 116 | 63-68 |
| DYNASAN® 118 | 72 |
| IMWITOR® 372 P | 62 |
| IMWITOR® 491 | 66-77 |
| IMWITOR® 900 K | 61 |
| SOFTISAN® 378 | 39 |
| WITEPSOL® E 85 | 43 |
| WITEPSOL® H32 | 32 |
| WITEPSOL® H16 | 34 |
| COMPRITOL® 888 ATO | 70 |
| COMPRITOL® HD5 ATO | 65-77 |
| PRECIROL® ATO 5 | 50-60 |
| H. VEG OIL | 55-65 |
| GV60 | 60-63 |
| VGB4 | 68-71 |
| VGB6 | 68-74 |
| VGB22 | 61-66 |
| VGB5ST | 69-73 |
| LAURIC ACID | 44 |
| BEES WAX | 60-64 |
| CARNAUBA WAX | 82 |
| COCONUT WAX | 30-39 |
| TRILAURIN | 47 |
| TRICAPRIN | 31-32 |
| POLYCAPROLACTONE | 60 |
| PALMITIC ACID | 63 |

List of nanoparticles that were tested: Silica, zinc oxide, clay, Magnesium hydroxide, Iron oxide, aluminum oxide, titanium dioxide, copper oxide, cellulose, starch, chitosan, etc. On the other hand, surface modified nanoparticles, by both physical and chemically modification ere tested and reported in the Example above

### SUMMARY OF THE FINDINGS

The formation of nano-shell microparticles was confirmed using scanning electron microscopy (SEM). The ability to form microparticles is dependent on the nanoparticle surface activity, the lipid core, the nanoparticle: lipid ratio; the preparation conditions (not shown).

TABLE 4:

particles according to the invention

| | | | | | | |
|---|---|---|---|---|---|---|
| Nanoparticle (NP) | Lipid (L) | NP:L | | | | |
| | | 1:50 | 1:20 | 1:10 | 1:5 | 1:2 |
| Cetyl alcohol | Silica | ++ | ++ | ++ | ++ | + |
| Cetyl alcohol | Silica-NH₂ | ++ | ++ | ++ | + | + |
| Cetyl alcohol | ZnO | - | - | - | ++ | ++ |
| Cetyl alcohol | CuO | + | - | - | ++ | - |
| Paraffin wax | Silica-NH₂ | ++ | ++ | ++ | ++ | ++ |
| Paraffin wax | Silica | - | - | - | - | - |
| Paraffin wax | TiO₂—NH₂ | ++ | ++ | ++ | ++ | + |
| Paraffin wax | Hydroxyapatite | - | - | ++ | ++ | ++ |
| Paraffin wax | ZnO | + | ++ | ++ | + | ++ |
| Paraffin wax | CuO | ++ | - | - | - | - |
| H. Veg oil | ZnO | - | + | ++ | ++ | ++ |
| H. Veg oil | CuO | - | - | - | - | ++ |
| H. Veg oil | Silica | - | - | - | - | - |
| H. Veg oil | Silica-NH₂ | ++ | ++ | ++ | ++ | ++ |
| Stearic acid | Silica | - | - | - | - | - |
| Witepsol H32 | Silica | - | - | - | - | - |

### Example 13: Microparticles With Multifunctional Surface Properties

The ability to embed nanoparticles of different properties allows obtaining microparticles of designed and desired properties. Nanoparticles of different properties, sizes and shapes are commercially available from different sources or can be synthesized using procedures published in common academic and technical reports have been used for making microparticles with different surface functionalities. Thus, for example, FePt magnetic nanoparticles (J. Nano Research Vol. 1 (2008) pp. 23-29) and Zn/Cu blend nanoparticles are used to prepare microparticles with a range of lipid cores, including: hydrogenated vegetable oil, cetyl alcohol, cetyl palmitate and solid paraffin. The particles are magnetic, antimicrobial and catalytic.

Nanoparticles containing surface polymerizable residues such as vinyl groups, amino and carboxylate groups, isocyanate groups are prepared by chemical bonding to particles surface groups such as hydroxyl group reacted with for example, diisocyanates, acryloyl or methacryloyl chloride, reactive silyl molecules and the like. These surface reactive groups can be polarized by light, heat or by radical polymerization. One application is in 3D printing inks where the printed ink is polymerized by light. Another application is polymerization after coating of a surface to improve adhesion and durability. Moreover, microparticles with metallic surfaces can be sintered or fused by heat or melt and the removal of the inner core by organic extraction, evaporation or degradation.

### Example 14: Microparticles With Nanoparticles of Different Sizes

Silica nanoparticles of the size: 20, 50, 100, and 600 nanometers were used to prepare microparticles with hydrogenated vegetable oil as core lipid. The particles were prepared using the method described in Examples 1-3. All particles were coated with phenylalanine prior to application. In all preparations, the weight ratio of nanoparticles to lipid cores remains constant as 1:10. The silica particles of 100 nm or below resulted in particles in the range of 20-50 microns in diameter while the larger microparticles yielded larger particles (>50 microns).

1-54. (canceled)

55. A particle comprising a solid lipid core, the circumference of the core being surface-decorated with a plurality of (nano)particles of at least one stabilizing material, the solid lipid core comprising or consisting at least one lipid-soluble or lipid-dispersible active agent, and wherein at least a portion of the (nano)particles comprising or associating to at least one active or non-active material, wherein the at least one lipid-soluble or lipid-dispersible active agent is same or different from said at least one active material.

56. The particle according to claim 55, wherein the solid lipid core having a size ranging from 0.3 to 200 microns.

57. The particle according to claim 55, wherein the lipid-core is free of nanoparticles.

58. The particle according to claim 55, wherein the density of the (nano)particles decorating the surface of the lipid core does not exceed 15%.

59. The particle according to claim 55, wherein the lipid-core comprises a solid lipid or water-insoluble solid polymer that is solid at a temperature between 25 and 100° C.

60. The particle according to claim 55, wherein the lipid core is of a material selected from a solid triglyceride, a wax fatty ester, a solid paraffin, a polymer, or a solid material capable of solubilizing or dispersing the lipid-soluble or water insoluble active agent.

61. The particle according to claim 55, wherein the lipid core consists the lipid-soluble or insoluble active agent.

62. The particle according to claim 55, wherein the lipid core is of a material selected from a wax, a beeswax, a paraffin wax, a hydrogenated vegetable oil, a long chain solid fatty alcohol and acid.

63. The particle according to claim 55, wherein the (nano)particles are particulate materials having a size (diameter) typically in the nanometer regime.

64. The particle according to claim 55, wherein the at least one lipid-soluble or lipid-dispersible active agent and said at least one active material, being same or different, are each independently selected pharmaceutical agents, veterinary agents, food components or additives, coloring agents and dyes, cosmetic agents, and agricultural agents.

65. The particle according to claim 64, wherein the at least one lipid-soluble or lipid-dispersible active agent and said at least one active material, being same or different, are each independently selected amongst pharmaceutical or veterinary agents.

66. The particle according to claim 65, wherein the agents are pharmaceutically acceptable agents.

67. The particle according to claim 66, wherein the pharmaceutically acceptable agent is oxybenzone, lidocaine, ibuprofen, ketoprofen or escitalopram.

68. The particle according to claim 55, wherein the active agent contained in at least a portion of the (nano)particles is a water soluble active agent, or an amphipathic active agent or a non-lipophilic agent.

69. A particle comprising a lipid core, the circumference of the core being surface-decorated with a plurality of (nano)particles of at least one stabilizing material, the lipid core being an active material or comprising at least one lipid-soluble or lipid-dispersible active material and wherein the (nano)particles optionally comprise or are associated to at least one active material.

70. A particle comprising a lipid core, the circumference of the core being surface-decorated with a plurality of (nano)particles of at least one stabilizing material, the lipid core being an active material or comprising at least one lipid-soluble or lipid-dispersible active material, wherein the lipid core is free or essentially free of nanoparticles, and wherein the plurality of (nano)particles optionally comprise or are associated to at least one active material.

71. An aqueous dispersion comprising a plurality of particles according to claim 55.

72. A product comprising at least one particle according to claim 55.

73. A method of preparing a dispersion according to claim 18, the method comprising mixing (i) a homogenous solution or a dispersion of at least one active agent in a melted lipid core material, and (ii) a dispersion of (nano)particles of a dispersing agent in an aqueous medium; under conditions permitting formation of the dispersion.

74. The method according to claim 73, wherein the homogenous solution or the dispersion is obtained by melting the lipid core material and dissolving (or dispersing) therein the at least one active agent.
